(19) United States
(12) Patent Application Publication
(10) Pub. No.: US 2015/0366820 A1
(43) Pub. Date: Dec. 24, 2015

(54) METHOD FOR PREVENTING AND/OR TREATING THE FORMATION OF INJURY OF LIVER AND ORAL COMPOSITION FOR USE IN SUCH METHOD

(71) Applicant: Flaxan GmbH & Co. KG, Nuremberg (DE)

(72) Inventor: Claus HELLERBRAND, Regensburg (DE)

(21) Appl. No.: 14/313,382

(22) Filed: Jun. 24, 2014

(51) Int. Cl. A61K 31/122

(52) U.S. Cl. CPC A61K 31/122

(57) ABSTRACT

A method for preventing and/or treating the formation of liver injury resulting from lipid accumulation in hepatocytes and/or inflammation of liver tissue and/or fibrosis in liver tissue and/or Non-Alcoholic Steatohepatitis (NASH) and/or alcoholic consumption and/or other toxic causes of liver injury by is performed by administering of iso-alpha-acid. An oral composition may be used.

This invention relates to a method for preventing and/or treating the formation of injury of the liver, in particular human liver, as well as to an oral composition for use in the prevention and/or treatment of liver injury, in particular human liver injury.

### BACKGROUND OF THE INVENTION

Liver injury and subsequent liver damage can result from plural causes such as infection with hepatitis B and hepatitis C viruses or alcohol abuse. The common pathology of all causes is that the liver injury is causing an inflammation of the liver (hepatitis) and a scaring reaction (fibrosis) of the liver tissue. The latter progresses to a complete fibrotic remodelling of the liver called cirrhosis. Cirrhosis is causing a loss of function of the liver and is associated with a high morbidity and mortality. Furthermore, liver inflammation and especially advanced fibrosis/cirrhosis are major risk factors for the development of liver cancer (hepatocellular cancer; HCC). Once, advanced liver damage/fibrosis has occurred it is irreversible i.e. cannot or only incompletely be cured anymore. Therefore, prevention of these advanced stages of liver damage is crucial.

In addition to the above mentioned causes, nowadays, liver injury also frequently results from “civilization factors” such as fatty and/or high caloric diet with or without lack of exercise and resulting obesity. These “civilization factors” cause a fat/lipid accumulation in the liver, i.e. a fatty liver (liver steatosis), which is also causing a liver injury. To distinguish this type of fatty liver from the fatty liver or liver disease caused by alcohol, it is called non-alcoholic fatty liver (non-alcoholic steatosis) or non-alcoholic fatty liver disease (NAFLD). The subsequent pathology of NAFLD is as described above, i.e. inflammation (in this case called: steatohepatitis), fibrosis, cirrhosis and liver cancer. Today, NAFLD is recognized as the most frequent liver disease worldwide and due to the increasing frequency of “civilization factors” this health problem will become even worse in the future. Hop (Humulus lupulus L.) has been originally known as a medical herb and has long been use for brewing of beer. Iso-alpha-acids (IAA) are derivatives or compounds produced through isomerization from alpha acids during the brewing process of beer (in particular during the step of boiling hops) and they are contained in beer as an active substance of bitter component. Iso-alpha-acid derivatives include isohumulone (formula I), isocohumulone (formula II) and isoadhumulone (formula III).

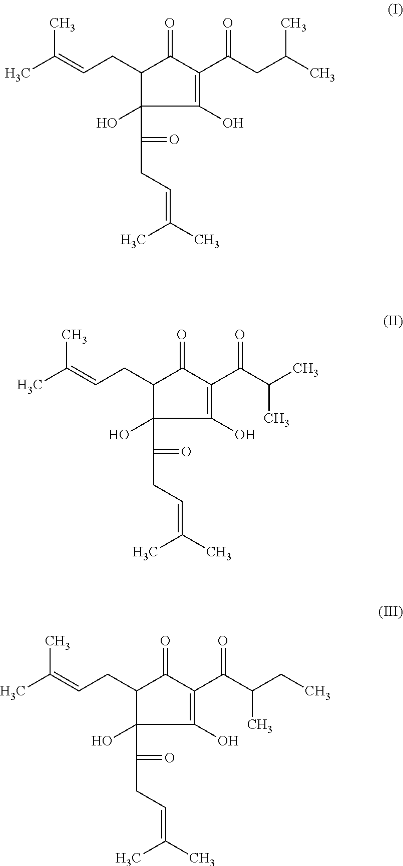

Iso-alpha-acids, as mentioned before, are produced by heating. There is no indication that IAA exhibits toxicity. Thus, iso-alpha-acids constitute a naturally derived compound without side risks. Iso-alpha-acids can be produced from a carbon dioxide-hop extract. Such method is disclosed e.g. in EP 0 474 892 B1.

The object of the present invention is to provide a new method and a new composition enabling an effective prevention or reduction of liver injury and liver damage.

### SUMMARY OF THE INVENTION

It has been found that liver injury and damage essentially resulting from

(a) lipid accumulation in hepatocytes and/or

(b) inflammation of liver tissue and/or

(c) fibrosis in liver tissue and/or

(d) Non-Alcoholic Steatohepatitis (NASH) and/or

(e) increased alcoholic consumption and/or

(f) other toxic causes of liver injury

can effectively be prevented and/or treated by administering iso-alpha-acid (IAA) Under the term “iso-alpha-acid” or “(IAA)” it is to be understood an iso-alpha-acid of one single structure or a mixture of iso-alpha-acids of different structure.

The administration of iso-alpha-acid has been found beneficial for an inhibition of the formation of an inflammation of liver tissue, of fibrosis or even of cancer which is a chain of causes. In case of fatty liver and NASH the administration of iso-alpha-acid inhibits the lipid accumulation of the liver which is the cause of fatty liver and NASH. Also in case of alcoholic liver injury, iso-alpha-acid inhibits lipid accumulation of the liver in addition to its beneficial effects on inflammation and fibrosis.

Administering iso-alpha-acid surprisingly reduces and/or even inhibits lipid accumulation in hepatocytes. Thus, inflammation caused by lipid accumulation in hepatocytes (fatty liver) can be prevented and/or treated by administering of iso-alpha-acid. Further subsequent stages of liver injury and damage or deterioration e.g. liver fibrosis, cirrhosis or even liver cancer can be avoided.

Liver inflammation caused by different mechanisms or different cell types in the liver, respectively, can be inhibited or even completely blocked. Thus, liver injury and damage can be inhibited in a very early phase of the disease.

In case of an initial formation of fibrosis in liver tissue such damaging process can be stopped by administering iso-alpha-acid. The liver as whole can recover.

Surprisingly, even the lipid accumulation in hepatocytes (steatosis) can be inhibited or completely blunted. Thus, in this type of liver injury (non-alcoholic fatty liver disease, NAFLD) not only the subsequent pathology but the actual cause of liver injury can be effectively inhibited or completely blocked and the development of subsequent liver injury, i.e. Non-Alcoholic Steatohepatitis (NASH), fibrosis, cirrhosis and liver cancer can be prevented.

Finally, liver injury resulting from increased alcoholic consumption, in particular alcohol induced fatty liver, i.e. alcoholic steatosis, inflammation of liver tissue and fibrosis can effectively be prevented and/or cured by administering of iso-alpha-acid. In particular, iso-alpha-acid given prophylacticly surprisingly prevents the formation liver injury and damage caused by alcohol.

In general, it is possible to administer iso-alpha-acid prophylacticly in order to prevent the appearance of the aforementioned pathologies of different causes of liver injury. Accordingly, iso-alpha-acid can be administered as an additive to nutrition over a long period of time as iso-alpha-acid do not have a toxic effect and can therefore be administered continuously for a longer time. This is most important for a prophylactic use as additive to nutrition.

Preferably, said iso-alpha-acid to be used is trans-iso-alpha-acid or cis-iso-alpha-acid.

Iso-alpha-acid is at least one selected from the group consisting of humulones, cohumulones, adhumulones, prehumulones, posthumulones or a combination thereof.

Said iso-alpha-acid is applied as a substance or compound derived i.e. prepared from the hop plant. However, also synthetically prepared iso-alpha-acids should be covered by this invention.

Iso-alpha-acid is preferably administered in a dosage of 0.01 to 30 mg/kg human body weight, preferably 0.5 to 10.0 mg/kg human body weight, most preferably 0.10 to 5.0 mg/kg human body weight. The lowest dosage should not be below 0.01 mg/kg human body weight. A dosage below 50.0 mg/kg human body weight is advantageous with respect to taste and bitterness of the iso-alpha-acid or a pharmaceutical form or dietary supplement containing iso-alpha-acid.

Iso-alpha-acid to be used should have a concentration in a range of 10-100%, preferably 20-80%, most preferably 30-60% by HPLC.

Said iso-alpha-acid is applied in the free acid form or in the salt form.

Preferably, said iso-alpha-acid can also be applied as an aqueous alkaline solution.

The present invention furthermore refers to an oral composition for use in the prevention and/or treatment of liver injury essentially resulting from

(a) lipid accumulation in hepatocytes and/or

(b) inflammation of liver tissue and/or

(c) fibrosis in liver tissue and/or

(d) Non-Alcoholic Steatohepatitis (NASH) and/or

(e) increased alcoholic consumption and/or

(f) other toxic causes of liver injury

said composition comprising essentially of a therapeutically effective amount of iso-alpha-acid, wherein the composition is in a dosage form for oral administration.

Preferably, said iso-alpha-acid is trans-iso-alpha-acid or cis-iso-alpha-acid.

Iso-alpha-acid is at least one selected from the group consisting of humulones, cohumulones, adhumulones, prehumulones, posthumulones or a combination thereof.

Iso-alpha-acid is applied as a substance or compound prepared from the hop plant.

Iso-alpha-acid is preferably administered in a dosage of 0.01 to 30.0 mg/kg human body weight, preferably 0.5 to 10.0 mg/kg human body weight, most preferably 0.1 to 5.0 mg/kg human body weight.

Iso-alpha-acid to be used should have purity in range of 10-100%, preferably 20-80%, most preferably 30-60%.

Said iso-alpha-acid is applied in the free acid form or in the salt form.

Preferably, said iso-alpha-acid can also be applied as an aqueous alkaline solution.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1A illustrates the beneficial effect of iso-alpha-acid (IAA) on lipid accumulation in primary human hepatocytes by illustration of hepatic triglyceride levels;

FIG. 1B illustrates the beneficial effect of IAA on lipid accumulation in primary human hepatocytes by illustration of photographs of lipid staining in cells.

FIG. 2 illustrates the beneficial effect of IAA on proinflammatory gene expression in human hepatic stellate cells based on analysis of MCP-1 expression.

FIG. 3 illustrates the beneficial effect of IAA on profibrogenic gene expression in human hepatic stellate cells based on analysis of COLL-1 expression;

FIG. 4A illustrates the beneficial effect of IAA in an in vivo model of acute alcohol-induced liver injury;

FIG. 4B shows a comparison between liver tissue of an alcohol-induced liver (ALc) and alcohol+IAA treated liver (ALc+IAA);

FIG. 5A illustrates the beneficial effect of IAA on liver injury and proinflammatory and profibrogenic gene in an in vivo model of acute liver injury induced by CCl₄; and

FIG. 5B illustrates the beneficial effect by illustration of photographs of liver injury by CCl₄ and CCl₄+IAA.

### DETAILED DESCRIPTION OF THE INVENTION

The invention provides a method for preventing and/or treating the formation of liver injury or damage resulting from lipid accumulation in hepatocytes and/or inflammation of liver tissue and/or fibrosis in liver tissue and/or Non-Alcoholic Steatohepatitis (NASH) and/or alcoholic consumption by administering of iso-alpha-acid. Furthermore the present invention provides an oral composition for use in the prevention and/or treatment of liver injury as aforementioned indicated. The present invention relates to the unexpected discovery that IAA is advantageous for preventing and/or curing liver injury and damage. Administering of IAA i.e. together with fatty diet or alcohol is beneficial as the damaging effect of a fatty diet as such and/or alcohol as such can be prevented or at least considerably be reduced.

IAA maybe administered as prophylactic means for the prevention of liver injury and also as means for treating i.e. curing liver injury.

The iso-alpha-acids to be used can be trans-iso-alpha-acid or cis-iso-alpha-acid. Said iso-alpha-acids are at least one selected from the group consisting of humulones, cohumulones, adhumulones, prehumulones and posthumulones or a combination thereof. Iso-alpha-acids are derivatives prepared from the hop plant. Iso-alpha-acids to be used according to the present invention have purity in range of 10-100%, preferably 20-80%, most preferably 30-60%.

Iso-alpha-acids may be applied in the free acid form or in the salt form.

Iso-alpha-acids can be administered in the form of tablets or capsules or added to food or beverages as dietary supplement.

Iso-alpha-acid is administered preferably in dosage of 0.01 to 30 mg/kg human body weight, preferably 0.5 to 10.0 mg/kg human body weight, most preferably 0.10 to 5.0 mg/kg human body weight.

The effects of the present invention shall be further illustratively explained by way of following examples.

### EXAMPLE 1

Example 1 reflects the effect of iso-alpha-acid on lipid accumulation in primary human hepatocytes

Example 1 is based in an in vitro model for liver steatosis as described in Wobser et al. (“Lipid accumulation in hepatocytes induces fibrogenic activation of hepatic stellate cells”, Cell Res. 2009 Aug; 19(8): 996-1005). It has been shown that pathological changes observed in this model do correctly simulate the pathological mechanism observed in human NAFLD.

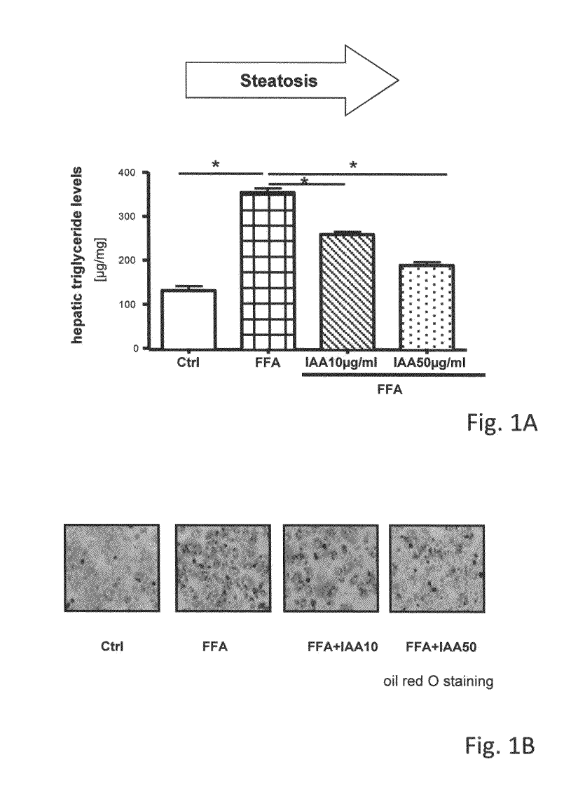

In the presence of free fatty acids (FFA) in the cell culture medium primary human hepatocytes become steatotic compared to control hepatocytes (CTRL) which are cultured without free fatty acids. In this model, IAA (Isohop® of Barth Haas) was added at different concentrations (IAA 10 mg/ml and IAA 50 mg/ml) to the cell culture medium together with FFA. Cellular steatotis is analysed by determination of cellular triglyceride content. As compared to CTRL hepatocytes FFA treated cells showed a significant increase of cellular triglyceride content (*: p<0.05). If IAA were added to the cell culture medium, one recognizes a dose dependent, significant (*: p<0.05) reduction of cellular steatosis compared to FFA without FFA as it is indicated in FIG. 1A.

This beneficial effect on cellular steatosis is also evident from photographs of the cell cultures stained with Oil red O as shown in FIG. 1B. Oil red O is a staining technique which detects cellular lipids by red colour. In accordance with the analysis of cellular triglyceride content, by raising the dosage of IAA the inhibitory effect of lipid accumulation can also be raised.

Together, both techniques clearly show that by administering IAA protection against liver steatosis can be realized.

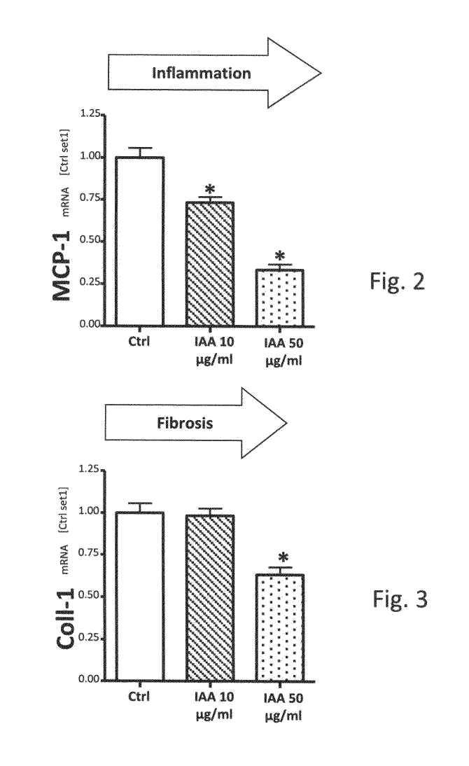

FIG. 2 shows a model of proinflammatory gene expression in human hepatic stellate cells (HSC). Once, these cells are an important source of proinflammatory factors upon liver injury and in liver disease, respectively. One of these factors is MCP-1, a chemokine which plays a central role in liver inflammation and fibrosis including fatty liver disease.

In this model the basal levels (control group) of MCP-1 mRNA expression in human hepatic stellate cells were analysed by quantitative PCR analysis.

Furthermore, MCP-1 expression was analyzed in HSC treated with different doses of IAA (24 hour stimulation). As shown, iso-alpha-acids inhibit dependent upon dosage the expression/formation of damaging pro inflammatory factor MCP-1.

FIG. 3

Hepatic stellate cells are also the effector cells of liver fibrosis, i.e. the cellular source of the fibrotic tissue, i.e. extracellular matrix (ECM) proteins. Collagen Type I (Coll-1) is quantitatively and qualitatively the most important ECM protein in liver fibrosis. In this model the basal levels (control group) of Coll-1 mRNA expression in human hepatic stellate cells were analysed by quantitative PCR analysis. Furthermore, Coll-1expression was analyzed in HSC treated with different doses of IAA (24 hour stimulation). As shown, iso-alpha-acids inhibit dependent upon dosage the expression/formation of the profibrogenic Coll-1.

It has to be mentioned that the expression of both factors (MCP-1 and Coll-1) in HSC and in liver injury, respectively, is regulated on the transcriptional level. Thus, mRNA levels adequately reflect the abundance of both pathological factors.

### EXAMPLE 2

Example 2 reflects a model of acute alcohol mediated liver injury, which was essentially described by Wagenberger (“Sex-specific differences in the development of acute alcohol-induced liver steatosis in mice”, Alcohol. 2013 November-December; 48(6): 648-56).

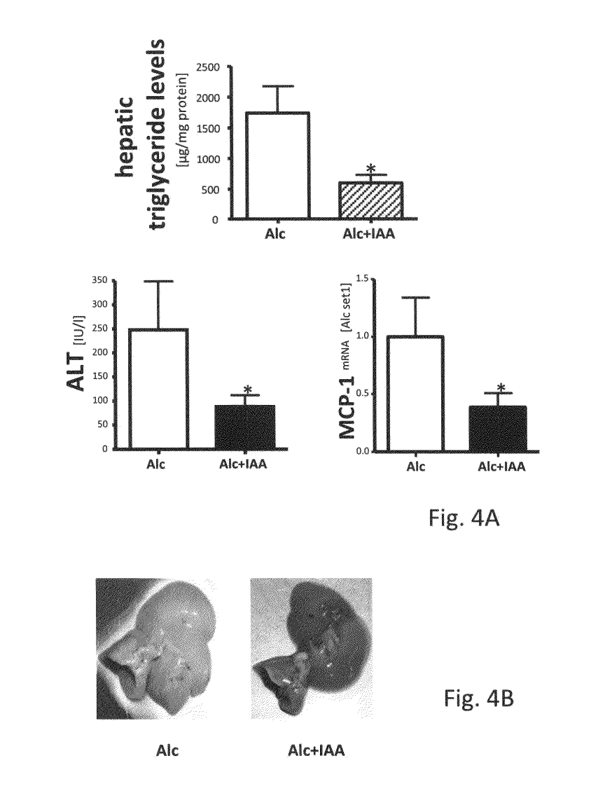

Alcohol was applied via gavage in a dose of 6 g/kg body weight (ALC group). The ALC+IAA group consisted of mice which received the alcohol together with IAA in the concentration as indicated. Each group consisted of 6 mice. As visible in the macroscopic image according to FIG. 4B ALc causes liver steatosis, which was almost invisible in the ALC+IAA group. Also hepatic tryglyceride levels, see FIG. 4A, as well as serum transaminases (ALT) were significantly lower in the ALC+IAA group. The same is true for the MCP-1.

### EXAMPLE 3

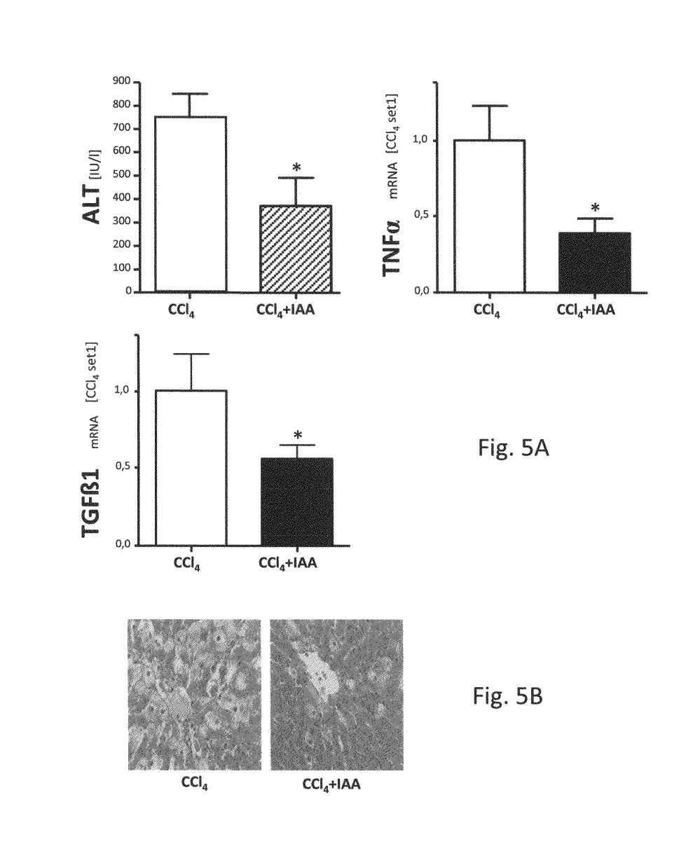

Example 3 refers to an in vivo model of acute liver injury by CCl₄. An acute liver injury in rats was induced by intraperitoneal application of tetrachlorcarbonate (CCl₄) representing a liver toxin. In a second group (CCl₄+IAA) the CCl₄ has been applied by gavage together with IAA. As compared to the CCI₄ group the serum transaminases (ALT) were significantly lower. TNF-alpha, a marker of liver inflammation, was significantly lower in the CCl₄-IAA group. Likewise, TGF-beta, a marker of liver fibrosis, was significantly lower in the CCl₄ -IAA group as illustrated in FIG. 5A. The images in FIG. 5B show that the liver histology does have less injury in case of CCl₄+IAA as in case of CCl₄. Thus, IAA deliver a beneficial effect on liver injury and proinflammatory and profibrogenic gene expression in the model of acute liver injury by CCl₄ in rats.

For all experiments Isohop® of Barth Haas has been used.

1. A method for preventing and/or treating formation of liver injury resulting from the group consisting of:
(a) lipid accumulation in hepatocytes,
(b) inflammation of liver tissue,
(c) fibrosis in liver tissue,
(d) Non-Alcoholic Steatohepatitis (NASH),
(e) alcoholic consumption, and
(f) other toxic causes of liver injury;
the method comprising:
administering iso-alpha-acid.

2. The method according to claim 1, wherein said iso-alpha-acid comprises trans-iso-alpha-acid.

3. The method according to claim 1, wherein said iso-alpha-acid comprises cis-iso-alpha-acid.

4. The method according to claim 1, wherein said iso-alpha-acid is at least one selected from the group consisting of: humulones, cohumulones, adhumulones, prehumulones, posthumulones or a combination thereof.

5. The method according to claim 1, comprising applying said iso-alpha-acid in enriched form prepared from a hop plant.

6. The method according to claim 1, comprising administering said iso-alpha-acid in a dosage of 0.1 to 5.0 mg/kg human body weight.

7. The method according to claim 1, wherein said iso-alpha-acid has a purity in a range of 30-60%.

8. The method according to claim 1, comprising applying said iso-alpha-acid in free acid form or in salt form.

9. The method according to claim 1, comprising applying said iso-alpha-acid as an aqueous alkaline solution.

10. An oral composition for use in the prevention and/or treatment of liver injury essentially resulting from:
(a) lipid accumulation in hepatocytes, and/or
(b) inflammation of liver tissue, and/or
(c) fibrosis in liver tissue, and/or
(d) Non-Alcoholic Steatohepatitis (NASH) and/or
(e) alcoholic consumption, and/or
(f) other toxic causes of liver injury;
said composition comprising a therapeutically effective amount of iso-alpha-acid, wherein the composition is in a dosage form for oral administration.

11. The composition according to claim 10, wherein said iso-alpha-comprises trans-iso-alpha-acid.

12. The composition according to claim 10, wherein said iso-alpha-acid comprises cis-iso-alpha-acid.

13. The composition according to claim 10, wherein said iso-alpha-acid is at least one selected from the group consisting of humulones, cohumulones, adhumulones, prehumulones, posthumulones or a combination thereof.

14. The method according to claim 10, wherein said iso-alpha-acid is applied in enriched form prepared from a hop plant.

15. The method according to claim 10, wherein said iso-alpha-acid has a purity in a range of 30-60%.

16. The method according to claim 10, wherein said iso-alpha-acid is applied in free acid form or in salt form.

17. The method according to claim 10, wherein said iso-alpha-acid is applied as an aqueous alkaline solution.
